(19) United States
(12) Patent Application Publication
(10) Pub. No.: US 2012/0296233 A9
(43) Pub. Date: Nov. 22, 2012

(54) Methods and apparatus for an analyte detecting device

(76) Inventor: Dominique M. Freeman, La Honda, CA (US)

(21) Appl. No.: 11/534,977

(22) Filed: Sep. 25, 2006

Related U.S. Application Data
(63) Continuation-in-part of application No. 10/237,261, filed on Sep. 5, 2002, now Pat. No. 7,344,507.
(63) Continuation of application No. 10/324,053, filed on Dec. 18, 2002, now Pat. No. 7,713,214.
(60) Provisional application No. 60/724,073, filed on Oct. 5, 2005.

(51) Int. Cl. A61B 5/00; B27N 3/08; B65B 55/00

(52) U.S. Cl. 600/583; 53/122; 264/239

(57) ABSTRACT

In one embodiment according to the present invention, a device is provided comprising a cartridge having a plurality of cavities. The device may include a plurality of penetrating members at least partially contained in the cavities of the single cartridge wherein the penetrating members are slidably movable to extend outward from lateral openings on the cartridge to penetrate tissue. The device may have a sterility barrier coupled to the cartridge, wherein the sterility barrier covers a plurality of the lateral openings, and wherein the sterility barrier covering the lateral openings is configured to be moved so that a penetrating member exits the lateral opening without contacting the barrier. The device may include a plurality of analyte detecting members coupled to the cartridge and a plurality of sample capture devices, wherein the sample capture devices each having an opening there through to allow a penetrating member to pass through.

### CROSS-REFERENCE TO RELATED APPLICATIONS

This application claims the benefit of U.S. Ser. No. 60/724,073, filed Oct. 05, 2005, which application is fully incorporated herein by reference.

### BACKGROUND OF THE INVENTION

1. Technical Field

The technical field relates to analyte detecting devices, and more specifically, coatings for improving glucose measurement.

2. Background Art

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Success rate generally encompasses the probability of producing a blood sample with one lancing action, which is sufficient in volume to perform the desired analytical test. The blood may appear spontaneously at the surface of the skin, or may be “milked” from the wound. Milking generally involves pressing the side of the digit, or in proximity of the wound to express the blood to the surface. In traditional methods, the blood droplet produced by the lancing action may reach the surface of the skin to be viable for testing.

When using existing methods, blood often flows from the cut blood vessels but is then trapped below the surface of the skin, forming a hematoma. In other instances, a wound is created, but no blood flows from the wound. In either case, the lancing process cannot be combined with the sample acquisition and testing step. Spontaneous blood droplet generation with current mechanical launching system varies between launcher types but on average it is about 50% of lancet strikes, which would be spontaneous. Otherwise milking is required to yield blood. Mechanical launchers are unlikely to provide the means for integrated sample acquisition and testing if one out of every two strikes does not yield a spontaneous blood sample.

Many diabetic patients (insulin dependent) are required to self-test for blood glucose levels five to six times daily. The large number of steps required in traditional methods of glucose testing ranging from lancing, to milking of blood, applying blood to the test strip, and getting the measurements from the test strip discourages many diabetic patients from testing their blood glucose levels as often as recommended. Tight control of plasma glucose through frequent testing is therefore mandatory for disease management. The pain associated with each lancing event further discourages patients from testing. Additionally, the wound channel left on the patient by known systems may also be of a size that discourages those who are active with their hands or who are worried about healing of those wound channels from testing their glucose levels.

Another problem frequently encountered by patients who may use lancing equipment to obtain and analyze blood samples is the amount of manual dexterity and hand-eye coordination required to properly operate the lancing and sample testing equipment due to retinopathies and neuropathies particularly, severe in elderly diabetic patients. For those patients, operating existing lancet and sample testing equipment can be a challenge. Once a blood droplet is created, that droplet must then be guided into a receiving channel of a small test strip or the like. If the sample placement on the strip is unsuccessful, repetition of the entire procedure including re-lancing the skin to obtain a new blood droplet is desired.

Early methods of using test strips required a relatively substantial volume of blood to obtain an accurate glucose measurement. This large blood requirement made the monitoring experience a painful one for the user since the user may need to lance deeper than comfortable to obtain sufficient blood generation. Alternatively, if insufficient blood is spontaneously generated, the user may need to “milk” the wound to squeeze enough blood to the skin surface. Neither method is desirable as they take additional user effort and may be painful. The discomfort and inconvenience associated with such lancing events may deter a user from testing their blood glucose levels in a rigorous manner sufficient to control their diabetes.

A further impediment to patient compliance is the amount of time that at lower volumes, it becomes even more important that blood or other fluid sample be directed to a measurement device without being wasted or spilled along the way. Known devices do not effectively handle the low sample volumes in an efficient manner Accordingly, improved sensing devices are desired to increase user compliance and reduce the hurdles associated with analyte measurement.

### SUMMARY OF THE INVENTION

An object of the present invention is to provide an analyte detecting apparatus that has improved measurement of analyte levels in a body fluid.

Another object of the present invention is to provide an improved method of manufacturing analyte detecting devices.

These and other objects of the present invention are achieved in a device that has a cartridge with a plurality of cavities. A plurality of penetrating members are at least partially contained in the cavities of the cartridge. The penetrating members are movable to extend outward from lateral openings on the cartridge to penetrate tissue. A sterility barrier is coupled to the cartridge. The sterility barrier covers the lateral openings and is at least partially movable to provide that a penetrating member exits the lateral opening without contacting the sterility barrier. A plurality of analyte detecting members are coupled to the cartridge. The analyte detecting members are associated with sample chambers. A plurality of sample capture devices are coupled to the sample chambers. The sample capture devices each have an opening to allow a penetrating member to pass through.

In another embodiment of the present invention, a method if provided of manufacturing an analyte detecting device. A cartridge is sized to fit within a housing. An opening is formed on the housing. At least one layer of viscoelastic material is applied on the housing around the opening. The material applies an compression force to a target tissue when the target tissue engages the material. A plurality of penetrating members are in the cartridge. A plurality of analyte detection devices are in the cartridge.

### BRIEF DESCRIPTION OF THE DRAWINGS

FIG. 1 illustrates an embodiment of a controllable force driver in the form of a cylindrical electric penetrating member driver using a coiled solenoid-type configuration.

FIG. 2A illustrates a displacement over time profile of a penetrating member driven by a harmonic spring/mass system.

FIG. 2B illustrates the velocity over time profile of a penetrating member driver by a harmonic spring/mass system.

FIG. 2C illustrates a displacement over time profile of an embodiment of a controllable force driver.

FIG. 2D illustrates a velocity over time profile of an embodiment of a controllable force driver.

FIG. 3 is a diagrammatic view illustrating a controlled feed-back loop.

FIG. 4 is a perspective view of a tissue penetration device having features of the invention.

FIG. 5 is an elevation view in partial longitudinal section of the tissue penetration device of FIG. 4.

FIG. 6 shows an exploded perspective view of one embodiment of a device according to the present invention.

FIG. 7 shows an exploded perspective view of a penetrating member cartridge.

FIG. 8 illustrates one embodiment of a cartridge that can be used the present invention.

FIG. 9 illustrates one embodiment of a sterility barrier of the present invention that covers the top of a disposable.

FIG. 10 illustrates one embodiment of electrical contacts to detecting members that can be used with the present invention.

FIG. 11 illustrates one embodiment of a penetrating member device of the present invention with a disposable disk.

FIG. 12 illustrates one embodiment of an instrument interface to the disposable of the present invention.

Referring now to FIG. 13, illustrates the concept of one microfluidic design embodiment of the present invention.

FIG. 14 illustrates one embodiment of sample capture elements used with the present invention.

FIG. 15 illustrates one embodiment of a pogo pin used in one embodiment of the present invention.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

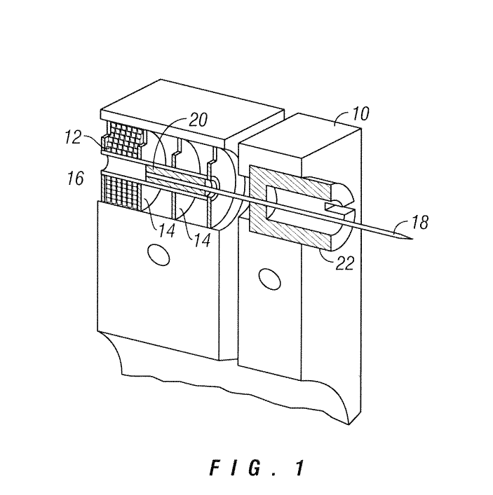

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 of a penetrating member device contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

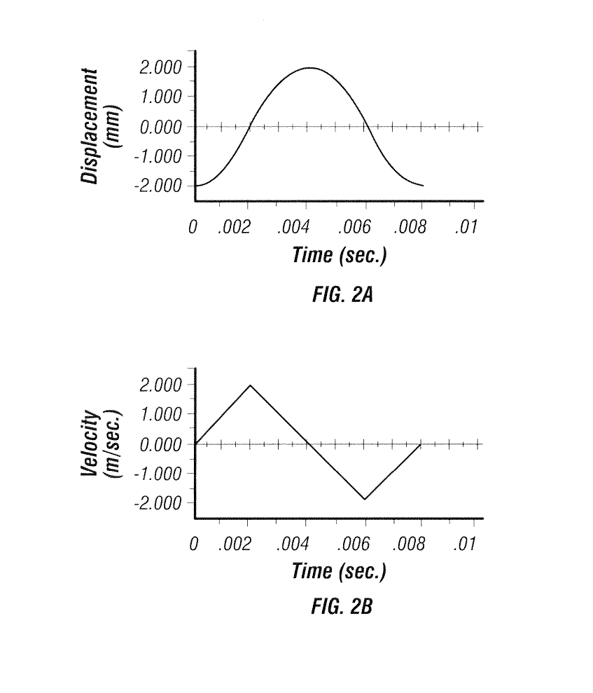

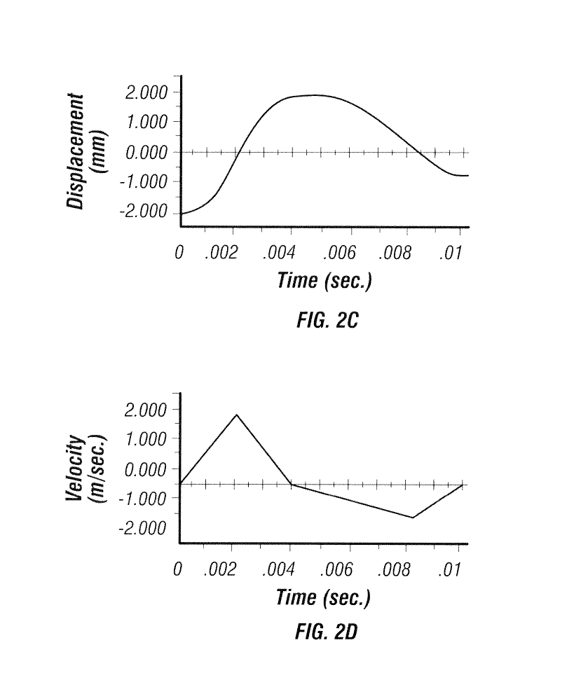

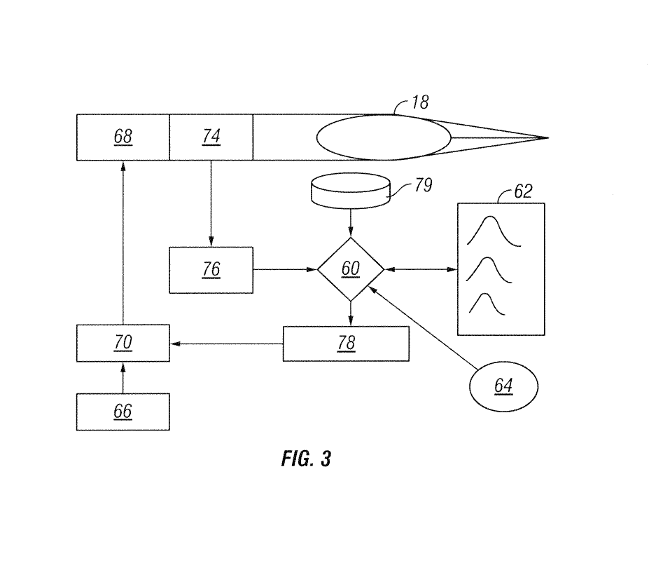

As discussed above, tissue penetration devices which employ spring or cam driving methods have a symmetrical or nearly symmetrical actuation displacement and velocity profiles on the advancement and retraction of the penetrating member as shown in FIGS. 2 and 3. In most of the available lancet devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2C which illustrates an embodiment of a controlled displacement profile and FIG. 2D which illustrates an embodiment of a the controlled velocity profile. These are compared to FIGS. 2A and 2B, which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of penetrating member, both diameter and penetrating member tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

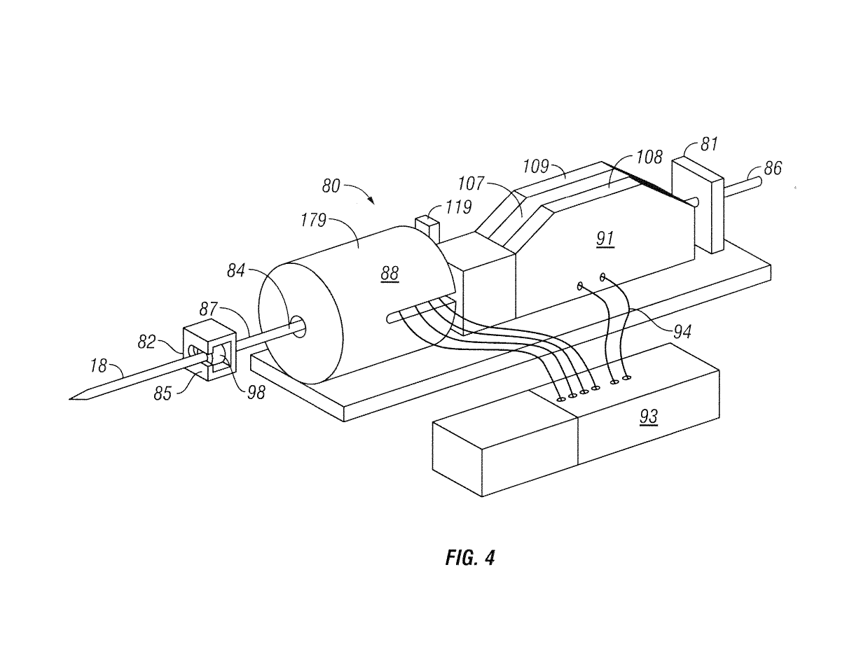

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

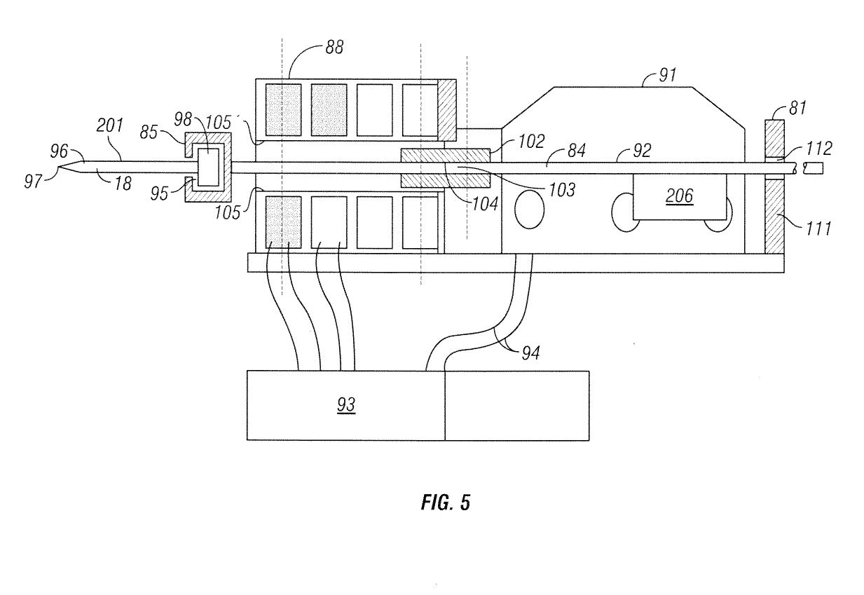

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91. The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

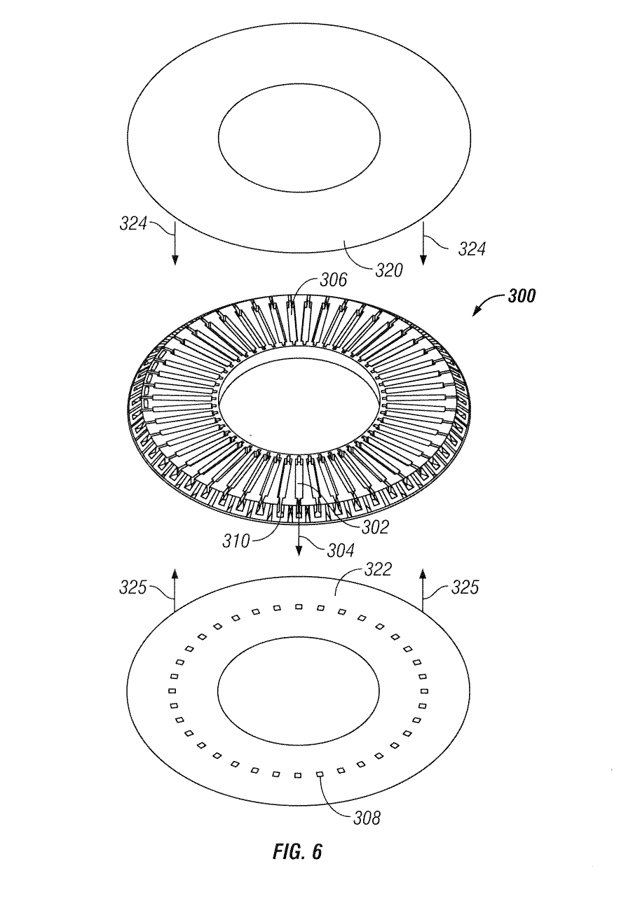

Referring now to FIG. 6, a still further embodiment of a cartridge according to the present invention will be described. FIG. 6 shows one embodiment of a cartridge 300 which may be removably inserted into an apparatus for driving penetrating members to pierce skin or tissue. The cartridge 300 has a plurality of penetrating members 302 that may be individually or otherwise selectively actuated so that the penetrating members 302 may extend outward from the cartridge, as indicated by arrow 304, to penetrate tissue. In the present embodiment, the cartridge 300 may be based on a flat disc with a number of penetrating members such as, but in no way limited to, (25, 50, 75, 100, . . . ) arranged radially on the disc or cartridge 800. It should be understood that although the cartridge 300 is shown as a disc or a disc-shaped housing, other shapes or configurations of the cartridge may also work without departing from the spirit of the present invention of placing a plurality of penetrating members to be engaged, singly or in some combination, by a penetrating member driver.

Each penetrating member 302 may be contained in a cavity 306 in the cartridge 300 with the penetrating member's sharpened end facing radially outward and may be in the same plane as that of the cartridge. The cavity 306 may be molded, pressed, forged, or otherwise formed in the cartridge. Although not limited in this manner, the ends of the cavities 306 may be divided into individual fingers (such as one for each cavity) on the outer periphery of the disc. The particular shape of each cavity 306 may be designed to suit the size or shape of the penetrating member therein or the amount of space desired for placement of the analyte detecting members 808. For example and not limitation, the cavity 306 may have a V-shaped cross-section, a U-shaped cross-section, C-shaped cross-section, a multi-level cross section or the other cross-sections. The opening 810 through which a penetrating member 302 may exit to penetrate tissue may also have a variety of shapes, such as but not limited to, a circular opening, a square or rectangular opening, a U-shaped opening, a narrow opening that only allows the penetrating member to pass, an opening with more clearance on the sides, a slit, a configuration as shown in FIG. 75, or the other shapes.

In this embodiment, after actuation, the penetrating member 302 is returned into the cartridge and may be held within the cartridge 300 in a manner so that it is not able to be used again. By way of example and not limitation, a used penetrating member may be returned into the cartridge and held by the launcher in position until the next lancing event. At the time of the next lancing, the launcher may disengage the used penetrating member with the cartridge 300 turned or indexed to the next clean penetrating member such that the cavity holding the used penetrating member is position so that it is not accessible to the user (i.e. turn away from a penetrating member exit opening). In some embodiments, the tip of a used penetrating member may be driven into a protective stop that hold the penetrating member in place after use. The cartridge 300 is replaceable with a new cartridge 300 once all the penetrating members have been used or at such other time or condition as deemed desirable by the user.

Referring still to the embodiment in FIG. 6, the cartridge 300 may provide sterile environments for penetrating members via seals, sterility barriers, covers, polymeric, or similar materials used to seal the cavities and provide enclosed areas for the penetrating members to rest in. In the present embodiment, a sterility barrier or seal layer 320 is applied to one surface of the cartridge 300. The seal layer 320 may be made of a variety of materials such as a metallic sterility barrier or other seal materials and may be of a tensile strength and other quality that may provide a sealed, sterile environment until the seal layer 320 is penetrate by a suitable or penetrating device providing a preselected or selected amount of force to open the sealed, sterile environment. Each cavity 306 may be individually sealed with a layer 320 in a manner such that the opening of one cavity does not interfere with the sterility in an adjacent or other cavity in the cartridge 800. As seen in the embodiment of FIG. 6, the seal layer 320 may be a planar material that is adhered to a top surface of the cartridge 800.

Depending on the orientation of the cartridge 300 in the penetrating member driver apparatus, the seal layer 320 may be on the top surface, side surface, bottom surface, or other positioned surface. For ease of illustration and discussion of the embodiment of FIG. 6, the layer 320 is placed on a top surface of the cartridge 800. The cavities 306 holding the penetrating members 302 are sealed on by the sterility barrier layer 320 and thus create the sterile environments for the penetrating members. The sterility barrier layer 320 may seal a plurality of cavities 306 or only a select number of cavities as desired.

In a still further feature of FIG. 6, the cartridge 300 may optionally include a plurality of analyte detecting members 308 on a substrate 822 which may be attached to a bottom surface of the cartridge 300. The substrate may be made of a material such as, but not limited to, a polymer, a sterility barrier, or other material suitable for attaching to a cartridge and holding the analyte detecting members 308. As seen in FIG. 6, the substrate 322 may hold a plurality of analyte detecting members, such as but not limited to, about 10-50, 50-100, or other combinations of analyte detecting members. This facilitates the assembly and integration of analyte detecting members 308 with cartridge 300. These analyte detecting members 308 may enable an integrated body fluid sampling system where the penetrating members 302 create a wound tract in a target tissue, which expresses body fluid that flows into the cartridge for analyte detection by at least one of the analyte detecting members 308. The substrate 322 may contain any number of analyte detecting members 308 suitable for detecting analytes in cartridge having a plurality of cavities 306. In one embodiment, many analyte detecting members 308 may be printed onto a single substrate 322 which is then adhered to the cartridge to facilitate manufacturing and simplify assembly. The analyte detecting members 308 may be electrochemical in nature. The analyte detecting members 308 may further contain enzymes, dyes, or other detectors which react when exposed to the desired analyte. Additionally, the analyte detecting members 308 may comprise of clear optical windows that allow light to pass into the body fluid for analyte analysis. The number, location, and type of analyte detecting member 308 may be varied as desired, based in part on the design of the cartridge, number of analytes to be measured, the need for analyte detecting member calibration, and the sensitivity of the analyte detecting members. If the cartridge 300 uses an analyte detecting member arrangement where the analyte detecting members are on a substrate attached to the bottom of the cartridge, there may be through holes (as shown in FIG. 76), wicking elements, capillary tube or other devices on the cartridge 300 to allow body fluid to flow from the cartridge to the analyte detecting members 308 for analysis. In other configurations, the analyte detecting members 308 may be printed, formed, or otherwise located directly in the cavities housing the penetrating members 302 or areas on the cartridge surface that receive blood after lancing.

The use of the seal layer 320 and substrate or analyte detecting member layer 822 may facilitate the manufacture of these cartridges 10. For example, a single seal layer 320 may be adhered, attached, or otherwise coupled to the cartridge 300 as indicated by arrows 324 to seal many of the cavities 306 at one time. A sheet 322 of analyte detecting members may also be adhered, attached, or otherwise coupled to the cartridge 300 as indicated by arrows 325 to provide many analyte detecting members on the cartridge at one time. During manufacturing of one embodiment of the present invention, the cartridge 300 may be loaded with penetrating members 302, sealed with layer 320 and a temporary layer (not shown) on the bottom where substrate 322 would later go, to provide a sealed environment for the penetrating members. This assembly with the temporary bottom layer is then taken to be sterilized. After sterilization, the assembly is taken to a clean room (or it may already be in a clear room or equivalent environment) where the temporary bottom layer is removed and the substrate 322 with analyte detecting members is coupled to the cartridge as shown in FIG. 6. This process allows for the sterile assembly of the cartridge with the penetrating members 302 using processes and/or temperatures that may degrade the accuracy or functionality of the analyte detecting members on substrate 322. As a nonlimiting example, the entire cartridge 300 may then be placed in a further sealed container such as a pouch, bag, plastic molded container, etc. . . . to facilitate contact, improve ruggedness, and/or allow for easier handling.

In some embodiments, more than one seal layer 320 may be used to seal the cavities 306. As examples of some embodiments, multiple layers may be placed over each cavity 306, half or some selected portion of the cavities may be sealed with one layer with the other half or selected portion of the cavities sealed with another sheet or layer, different shaped cavities may use different seal layer, or the like. The seal layer 320 may have different physical properties, such as those covering the penetrating members 302 near the end of the cartridge may have a different color such as red to indicate to the user (if visually inspectable) that the user is down to say 10, 5, or other number of penetrating members before the cartridge should be changed out.

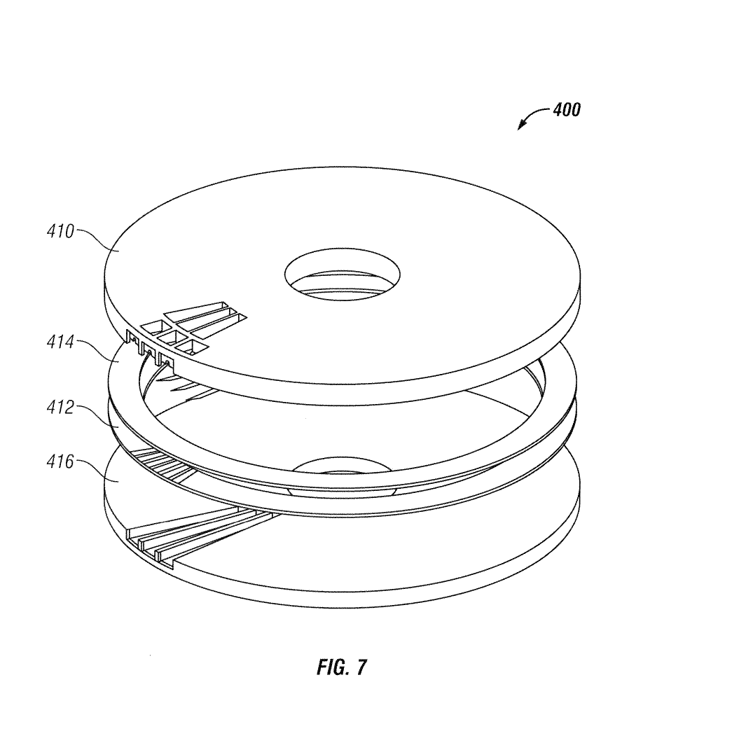

Referring now to FIG. 7, another embodiment of the present invention will now be described. The cartridge 400 is a fully integrated sampling/measurement solution is comprised of an integrated sampling/measurement disposable, and an electronic blood-sampling device embedded within a glucose measurement instrument.

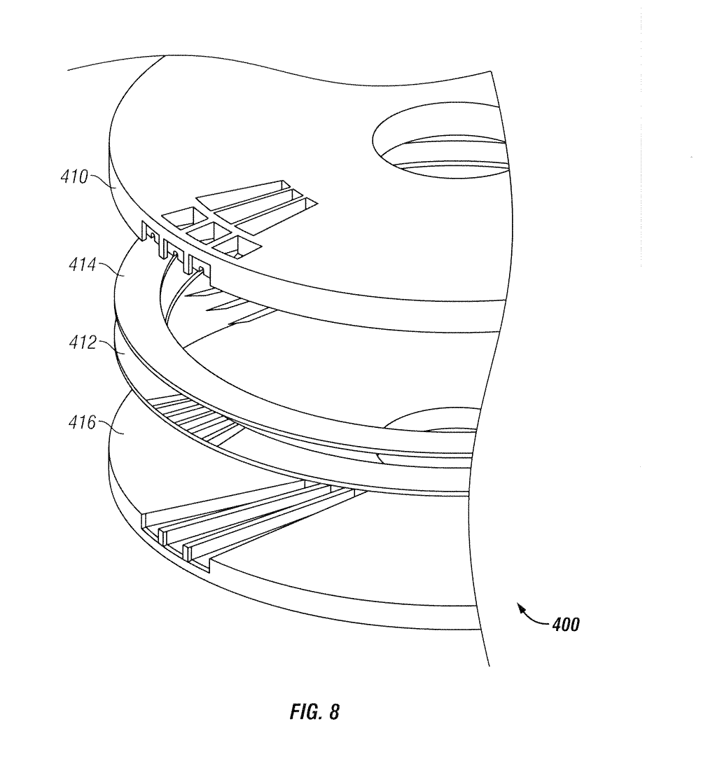

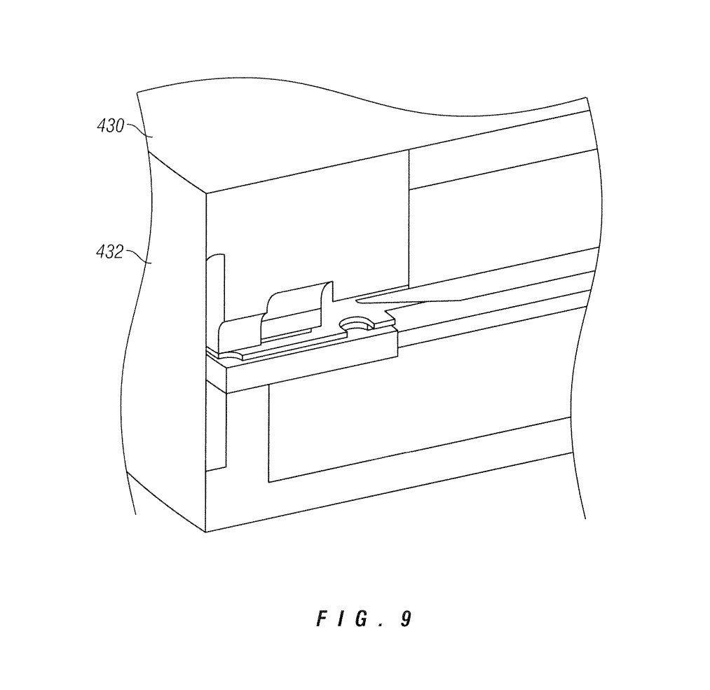

FIG. 8 shows the cartridge 400 in more detail. FIG. 8 shows that the cartridge 400 is comprised of an penetrating member disk 410 with the glucose detecting members attached on the bottom of the disk. The penetrating member passes over the top of the detecting member rather than through the detecting member substrate. Sample capture may be facilitated by microfluidic structures at the circumferential edge of the disk.

The current preferred embodiment for cartridge 400 came about through discussions about how to solve the sealing for moisture protection challenge as well as an alternative to the through hole connector solution. The general configuration for sample capture and detecting member fill and attachment of the detecting member ring are shown. The paradigm of a 50 penetrating member disposable with a single molded support is simple compared to other solutions where 50 penetrating members bearing 50 molded chucks and sterility caps are placed in a disk, drum or bandolier, and the number of moving parts can exceed the number of penetrating members or tests in general!

Extending the penetrating member disk 410 idea and attaching a 50-detecting member ring to the bottom of the 50 penetrating member disk seems an appropriate within the context of the penetrating member lancing device paradigm, though it requires a shift from the current premise of packaging single detecting members into a disposable carrying 50 single detecting members for which the industry seems more prepared to manufacture.

Components

FIGS. 7 and 8 show one embodiment of a cartridge 400. The disposable consists of an penetrating member lancing device molded disks to which is bonded a ring of detecting members. The detecting member ring 412 and penetrating member disk 410 are separated by a laminate structure 414 that is configured to guide blood from the sample inlet port into the detecting member. Below the GlucoSens ring is a desiccant disk 416, which contains a molded desiccant.

As seen in FIG. 9, the sterility barrier 430 covers the disposable on the top (as the current penetrating member disk 410) and on the front 432 though the front surface foiling is not angled to cover the chamfered edge as in the penetrating member lancing device. The current punch and plough configurations have been deemed workable for the Titan that has similar front punch requirements to remove the sterility barrier from the disposable, analyte sensor support ring. A side view of the disposable reveals the relationship between the laminate structure and the detecting member as well as the “stop arch” included to prevent excess blood following the penetrating member back into the penetrating member channel. The current volume requirement of this design with the Huygens sample capture is about 150 nL.

In one embodiment of the present invention, a cartridge is provided with a plurality of cavities. A plurality of penetrating members are at least partially contained in the cavities of the cartridge. The penetrating members are movable to extend outward from lateral openings on the cartridge to penetrate tissue. A sterility barrier is coupled to the cartridge. The sterility barrier covers the lateral openings and is at least partially movable to provide that a penetrating member exits the lateral opening without contacting the sterility barrier. A plurality of analyte detecting members are coupled to the cartridge. The analyte detecting members are associated with sample chambers. A plurality of sample capture devices are coupled to the sample chambers. The sample capture devices each have an opening to allow a penetrating member to pass through.

In another embodiment of the present invention, a method if provided of manufacturing an analyte detecting device. A cartridge is sized to fit within a housing. An opening is formed on the housing. At least one layer of viscoelastic material is applied on the housing around the opening. The material applies an compression force to a target tissue when the target tissue engages the material. A plurality of penetrating members are in the cartridge. A plurality of analyte detection devices are in the cartridge.

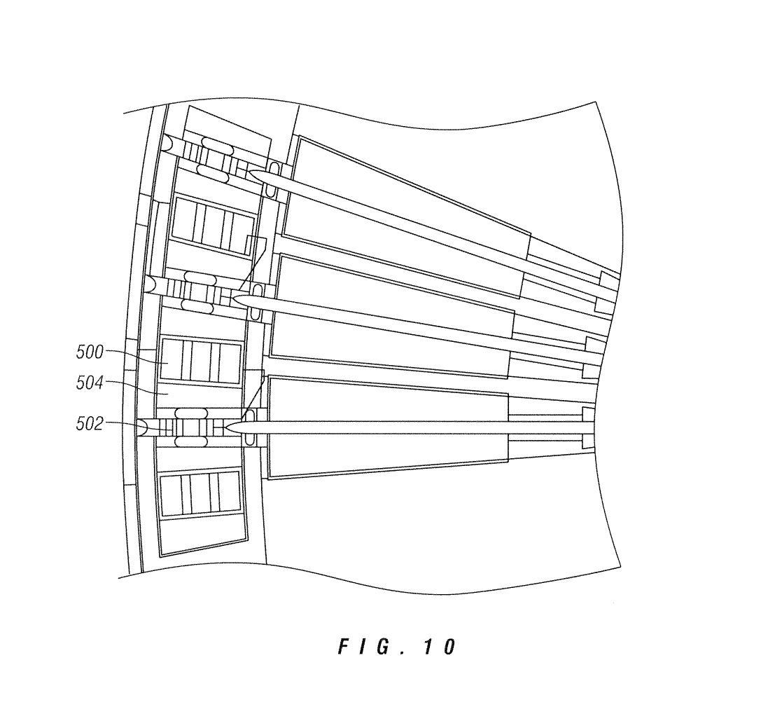

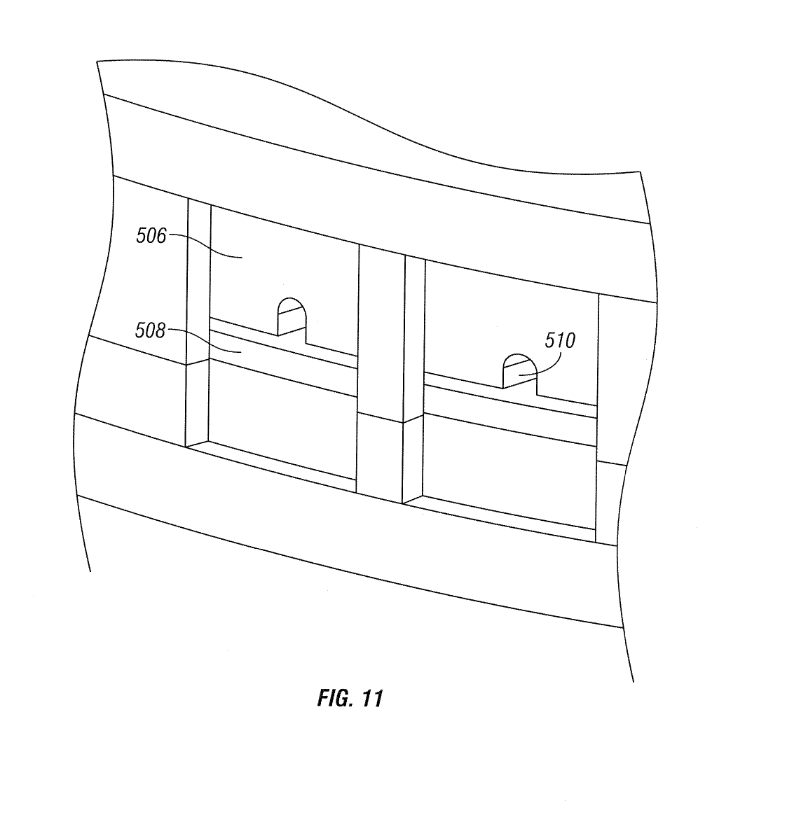

In one embodiment as seen in FIG. 10, electrical contacts to the detecting members will be made through the top using the miniaturized pogo pin described in detail in the section “connectors”. Details of the top view show the connector trenches as well as the sealing area between the detecting members.

The detecting members will be screen-printed the same as in the current method up to the level of the hydrophobic PSA. The laminate film containing the “rib” features will be aligned and pressed on to the detecting member. The ribs (or slits) will be laser cut, and the laminate aligned to screen-printed features. The density of the detecting members per sheet has been estimated for 120°, 180° and 360° arcs. Comparable packing densities are achievable because the “handle” of the lollipop is not needed (this function is provided by the laminate which is inside (and forms a part) the detecting member channel, and there is no longer a space needed between every detecting member for cuffing/punching into individual “Chiclets”. The 120° nested layout produces the most detecting member per sheet at 2132. The challenge will be to connect the arcs into a single ring for assembly on to the penetrating member disk 410.

Functional Performance

Referring now to FIG. 11, in one embodiment the features present in the penetrating member lancing device disposable used for indexing, gripping, firing and main punching activities. The penetrating member disk is an upside down version of the penetrating member lancing device disposable, the same footprint (57 mm) and the total thickness (with detecting member ring) is 4.8 mm. The main pocket dimensions remain unchanged, and there is no change in the maximum displacement of 3.7 mm. The rear bearing remains the same as well as the pinch. A “V” notch above the penetrating member creates the front bearing and a screen-printed or embedded pad on the laminate.

The front face is designed to be opened by the plough method, which currently the preferred method for Titan. The front dace will be sealed with foil, probably using the radial heat seal rig. The finger may contact the ring inside of the front face window, which may require some design optimization for correct finger placement. Work on the plough design might achieve this; conversely some features on the sample inlet aperture may also address the problem of correct finger positioning to capture blood from the wound.

Instrument Interface

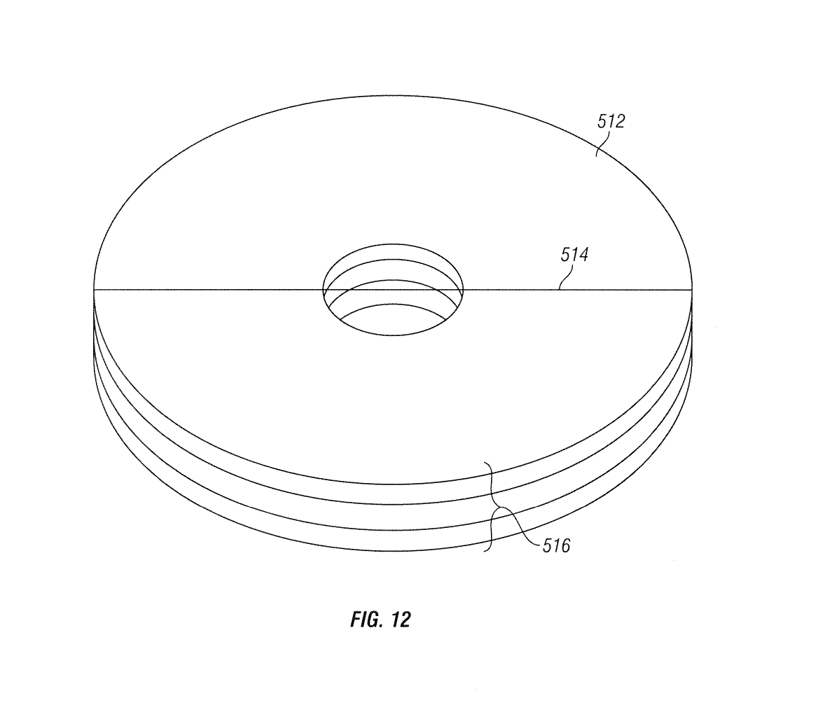

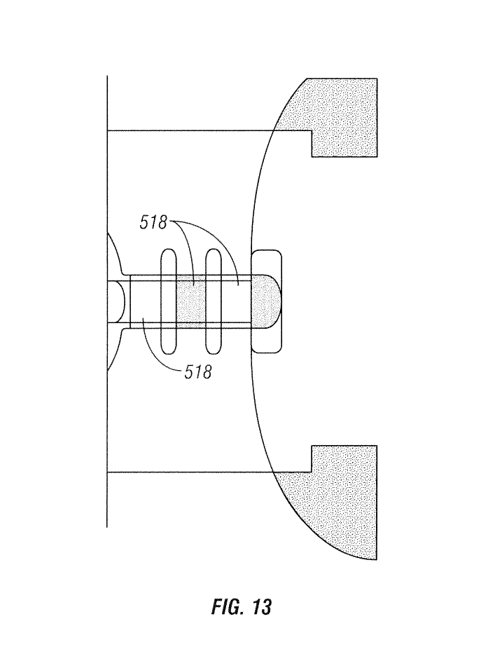

Referring now to FIG. 12, the instrument interface to the disposable will require a bar code on the top. The advantage is that the main punch, electrical connector access is on the instrument side allowing for a slim door. As a comparison, the Titan dimensions are 61 mm in diameter, 9 mm thick.

Sample Capture

A two-pronged approach to investigating a sample capture strategy for cartridge 400 was employed, lab experiments using a test rig of the preferred embodiment and theoretical simulation of same. The object is to identify the geometry best suited for the filling of the sample channel and to discover potential risks that may influence the desired filling.

Preferred embodiments of sample capture and detecting member fill configurations were derived at a workshop held in Toft on Apr. 26-27 2005 ADX-0028-D-A Ecoburger Phase 1 Sample capture, detecting member layout and sealing. From the workshop three major concepts for sample capture and detecting member layout were chosen for testing and simulation, as they were deemed best suited to rapid development.

In A and B the disposable has no sample capture structure. In A the glucose detecting member is placed in the penetrating member tunnel and relies on the penetrating member acting as the cover slip. In B (Bravo) three “ribs” cover the detecting member and opposed to one “rib” in E (Echo). C (Charlie) has the sample capture structure of the Titan “Huygens” detecting member with a three rib configuration. D was considered high risk and not pursued.

Referring now to FIG. 13, a microfluidic design embodiment of the present invention was used for development of the construction and testing methods. The end result of each test was deduced from examination under a microscope, as the videos were not easily decipherable Photos taken using the microscope are included in the detailed report and are too numerous to include here. Some smearing was encountered due to the fact that blood was brought to the aperture after lancing. In addition clamp force was important in preventing microcapillarities forming between the layers and wicking blood away from the main channel.

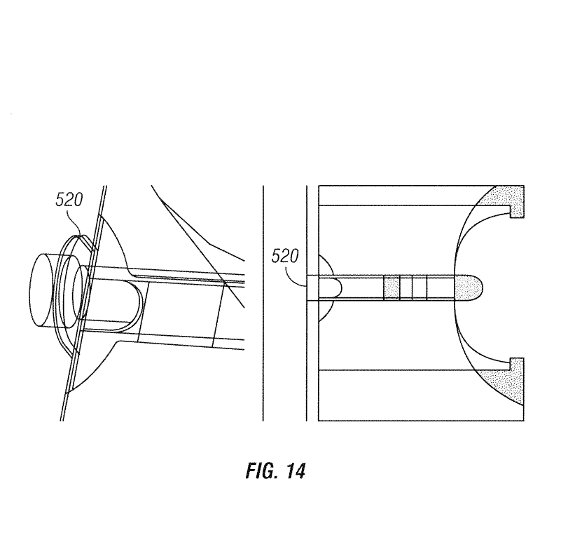

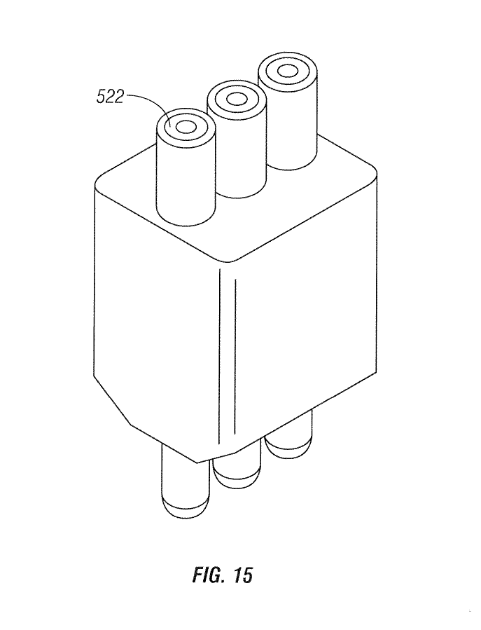

FIG. 14 shows another embodiment of a sample capture elements used with the present invention. The sample capture structures may be overlayed on the analyte detecting member.

FIG. 15 shows one embodiment of a pogo pin used to obviate the use of holes through the disposable to provide connectivity. The contact pads would be connected using a small pogo pin connector. This embodiment may be a 0.9 custom pitch pad. The contact pads are within the top sterility barrier covering to minimize moisture ingress, and this is punched prior to use. The sterility barrier would not be able to contact the pogo pins and short-circuit them.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the dissolvable seal may or may not be included.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. U.S. Provisional applications Ser. Nos. 60/610,305, 60/610,360,and 60/611,094 are fully incorporated herein by reference for all purposes.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A device comprising;
a cartridge having a plurality of cavities;
a plurality of penetrating members at least partially contained in said cavities of the cartridge, the penetrating members being movable to extend outward from lateral openings on said cartridge to penetrate tissue;
a sterility barrier coupled to said cartridge, said sterility barrier covering the lateral openings and at least partially movable to provide that a penetrating member exits the lateral opening without contacting the sterility barrier;
a plurality of analyte detecting members coupled to said cartridge, the analyte detecting members being associated sample chambers; and
a plurality of sample capture devices coupled to the sample chambers, said sample capture devices each having an opening there through to allow a penetrating member to pass through.

2. The device of claim 1, wherein each of a penetrating member has a tip that is not physically connected to the sterility barrier, the sterility barrier covering the lateral openings being configured to be moved so that a penetrating member exits the lateral opening without contacting the barrier.

3. The device of claim 1, wherein the sterility barrier covering the lateral openings is configured to be moved substantially vertically so that a penetrating member exits the lateral opening without contacting the barrier.

4. The device of claim 1, wherein the sterility barrier covering the lateral openings is configured to be punched downward so that a penetrating member exits the lateral opening without contacting the barrier.

5. The device of claim 1, wherein the sterility barrier covering the lateral openings at least partially breaks away so that a penetrating member exits the lateral opening without contacting the barrier.

6. The device of claim 1, wherein the sterility barrier is positioned to define a surface at an angle between about 3 degrees and 90 degrees, relative to horizontal.

7. The device of claim 1, wherein the sterility barrier is positioned to define a surface at an angle of about 45 degrees, relative to horizontal.

8. The device of claim 1, wherein the sterility barrier is made of a material containing one of the following: aluminum, polymer, and paper.

9. The device of claim 1, wherein the sterility barrier is made of a laminate made from one of, aluminum, polymer, and paper.

10. The device of claim 1 wherein each of said sample capture devices includes a rib oriented with a longitudinal axis generally perpendicular to a line of travel of the penetrating member.

11. The device of claim 1 wherein each of said sample capture devices includes a plurality of ribs, each oriented with a longitudinal axis perpendicular to the line of travel of the penetrating member, said ribs located over the analyte detecting member.

12. The device of claim 1 wherein said sample capture devices formed on a ribbon or tape structure.

13. The device of claim 1 wherein said sample capture devices are formed on circular disc and coupled to the cartridge.

14. The device of claim 1 wherein said sample capture devices each includes a wicking member.

15. The device of claim 1 wherein said sample capture devices each includes a hydrophilic membrane.

16. The device of claim 1 wherein said sample capture devices each includes a wicking member and a capillary member.

17. The device of claim 1 wherein said sample capture devices each has a lollipop shaped opening on the vertical portion.

18. The device of claim 1 wherein said sample capture devices are each individually movable to be in a valve open or a valve closed position.

19. The device of claim 1 wherein said sample capture devices are each hinged to a disc.

20. A method of manufacturing an analyte detecting device, said method comprising:
providing a housing;
providing a cartridge that is sized to fit within the housing;
forming an opening on said housing;
applying at least one layer of viscoelastic material on said housing around said opening, said material applying an compression force to a target tissue when the target tissue engages said material;
providing a plurality of penetrating members in said cartridge; and
providing a plurality of analyte detection devices in said cartridge.

21. The method of claim 20, wherein each of a penetrating member has a tip that is not physically connected to the sterility barrier, the sterility barrier covering the lateral openings being configured to be moved so that a penetrating member exits the lateral opening without contacting the barrier.

22. The method of claim 20, wherein the sterility barrier covering the lateral openings is configured to be moved substantially vertically so that a penetrating member exits the lateral opening without contacting the barrier.

23. The method of claim 20, wherein the sterility barrier covering the lateral openings is configured to be punched downward so that a penetrating member exits the lateral opening without contacting the barrier.

24. The method of claim 20, wherein the sterility barrier covering the lateral openings at least partially breaks away so that a penetrating member exits the lateral opening without contacting the barrier.

25. The method of claim 20, wherein the sterility barrier is positioned to define a surface at an angle between about 3 degrees and 90 degrees, relative to horizontal.

26. The method of claim 20, wherein the sterility barrier is positioned to define a surface at an angle of about 45 degrees, relative to horizontal.

27. The method of claim 20, wherein the sterility barrier is made of a material containing one of the following: aluminum, polymer, and paper.

28. The method of claim 20, wherein the sterility barrier is made of a laminate made from one of, aluminum, polymer, and paper.

29. The method of claim 20, wherein said analyte detection devices measure glucose levels in a sample fluid.

30. The method of claim 20, wherein said penetrating members are lancets.

31. The method of claim 20, further comprising placing a solenoid penetrating member driver in said housing.

32. The method of claim 20, wherein said viscoelastic material is formed in the shape of an O-ring.

33. The method of claim 20, wherein said viscoelastic material is formed to have a primary portion and a secondary portion, said secondary portion engaging a portion of the tissue away from a lancing site and compressing the tissue to drive blood towards the lancing site.

34. The method of claim 20, further comprising providing a plurality of seals on said cartridge to maintain said penetrating members in a sterile condition prior to use.

35. The method of claim 20, further comprising providing an LCD screen on said housing to provide information to the user during use.

36. The method of claim 20, further comprising providing an LCD screen on said housing to provide information to the user during use.
